(19) United States
(12) Patent Application Publication
(10) Pub. No.: US 2003/0198693 A1
(43) Pub. Date: Oct. 23, 2003

(54) Home-made nutraceutical capsules and tablets

(76) Inventor: Eli Pinthus, Maale Adumim (IL)

Correspondence Address: BROWDY AND NEIMARK, P.L.L.C., 624 NINTH STREET, NW, SUITE 300, WASHINGTON, DC, 20001-5303, US

(21) Appl. No.: 10/124,762

(22) Filed: Apr. 18, 2002

(51) Int. Cl. A61K 35/78; A61K 9/48

(52) U.S. Cl. 424/725; 424/451

(57) ABSTRACT

A method and apparatus for self-production of capsules and tablets comprising nutraceuticals is described. The method comprises of receiving appropriate instructions, raw materials and the appropriate apparatus for either capsulating or tableting. Tablets and capsules comprising the desired constituents of nutraceuticals are obtained.

### FIELD OF THE INVENTION

This invention relates to the production of nutraceuticals. In particular it relates to a method for self-production of nutraceuticals.

### BACKGROUND OF THE INVENTION

Dietary supplements in the form of vitamins, minerals and natural products claimed to regulate metabolism are widely used. These supplements are termed nutraceuticals, and their consumption is growing steadily in recent years. Total sales volume exceeded 20B$ in 2001. Over 4B$ sales are generated only from capsules and tablets which are sold in retail chains, health shops etc. The current annual growth rate is estimated at about 4%, lower than that of a growth of about 15-20% per annum in previous years. Thus growth is stagnating.

Consumer awareness to the consumption of nutraceuticals is growing. Market data published recently reported that over 50% of the US population are defined as nutraceutical consumers. 25% of these are purchasing those products on regular basis. Over 100 companies are involved in production and marketing of large variety of final nutraceutical products.

With the advancement of flow and availability of data (Internet and popular scientific articles), the profile of consumption becomes more individual. Consumers attempt to build their own individual list of nutraceuticals, which fits best to their needs according to their understanding. In many cases an individual can not find a single commercial product meeting his needs. Only a combination of commercial products may satisfy the individual consumer. It should be noted that the wholesale raw material cost presents in most cases no more than about 15% of the retail price. However, due to huge marketing and promotion costs, the final retail prices of the products are relatively high, and combining several commercial products may be a costly solution. There is thus a need to develop a method for bridging the gap between the commercial products and the individual quests.

### SUMMARY OF THE INVENTION

The present invention is directed to a commercially available kit, for a feasible production of nutraceutical capsules or tablets in a personalized manner for meeting each individual needs and desires.

Thus the present invention is directed to a kit designed for self-producing capsules or tablets containing at least one nutraceutical. The kit for self-producing of capsules comprises:

(i) a system operable to

receiving user selected at least one nutraceutical ingredient, wherein said at least one nutraceutical ingredient is in a pre-defined quantity;

receiving plurality of empty capsules;

pouring said at least one nutraceutical ingredient into said plurality of empty capsules, giving rise to filled capsules; and

closing said filled capsules, giving rise to personalized capsules;

(ii) instructions for the preparation of the personalized capsules including designation of the pre-defined quantity of said at least one nutraceutical ingredients.

The kit for self-producing of tablets comprises:

(i) a system operable to

receiving user selected at least one nutraceutical ingredient, wherein said at least one nutraceutical ingredient is in a pre-defined quantity;

receiving at least one excipient;

pouring said at least one nutraceutical ingredient and at least one excipient into a feeder of a tableting apparatus;

pressing a predetermined quantity of said at least one nutraceutical ingredient together with at least one excipient in said tableting apparatus, giving rise to personalized tablet; and

repeating said pouring and pressing steps giving rise to personalized tablets;

(ii) instructions for the preparation of tie personalized tablets including designation of the pre-defined quantity of said at least one nutraceutical ingredient and said at least one excipient.

The various nutraceuticals, auxiliary—nonactive components and the empty capsules are purchased from a local distributor, where the nutraceutical ingredient in the case of tables may include at least one pre-defined quantity of excipient.

The invention is further directed to a method for self-producing of personalized capsules or tablets containing at least one nutraceutical ingredient. In the case of capsules the methods comprises:

providing instructions for the quantity required for producing a user selected capsule comprising at least one nutraceutical ingredient;

providing instructions how to self-produce personalized capsules, including:

(i) receiving plurality of empty capsules;

(ii) pouring said at least one nutraceutical ingredient into said plurality of empty capsules, giving rise to filled capsules; and

(ii) closing said filled capsules, giving rise to personalized capsules.

In the case of tablets the methods comprises:

providing instructions for the quantity required for producing a user selected tablet comprising at least one nutraceutical ingredient;

providing instructions how to self-produce personalized tablets, including:

(i) receiving at least one excipient;

(ii) pouring said at least one nutraceutical ingredient and at least one excipient in a predefined quantity into a feeder of a tableting apparatus;

(iii) pressing a predetermined quantity of said at least one nutraceutical ingredient together with at least one excipient in said tableting apparatus, giving rise to personalized tablet; and

(iv) repeating said pouring and pressing steps giving rise to personalized tablets.

It should be noted that the nutraceutical ingredient may already comprise a predefined quantity of at least one excipient required for tableting.

The invention is further directed to at least one nutraceutical ingredient and associated instructions for use in a process for self-producing of personalized capsules, the instructions comprising:

initial instructions for the maximum quantity per capsule of said at least one nutraceutical ingredient to use in the process; the process comprising:

(i) receiving plurality of empty capsules;

(ii) pouring said at least one nutraceutical ingredient into said plurality of empty capsules, giving rise to filled capsules; and

(iii) closing said filled capsules, giving rise to personalized capsules.

The is yet further directed to at least one nutraceutical ingredient and associated instructions for use in a system of self-producing of personalized tablets, the instructions comprising:

initial instructions for the maximum quantity per capsule of said at least one nutraceutical ingredient and said at least one excipient to use in the process; the process comprising:

(i) receiving at least one excipient;

(ii) pouring said at least one nutraceutical ingredient and at least one excipient into a feeder of a tableting apparatus;

(iii) pressing a predetermined quantity of said at least one nutraceutical ingredient together with at least one excipient in said tableting apparatus, giving rise to personalized tablet; and

(iv) repeating said pouring and pressing steps for a desired number of cycles giving rise to personalized tablets.

The invention is yet further directed to a method of self-producing capsules or tablets, where a person desirous of obtaining personalized tablets or capsules comprising of at least one desired nutraceutical ingredient may submit the order to a local distributor. The distributor would then provide the desirous person with the appropriate instructions and required materials for such self-production.

The invention in addition is further directed to an apparatus for self-producing of tablets. The apparatus is equipped with an opening for receiving the raw material comprising the ingredients of a tablet and appropriate means for compressing the raw material into a tablet.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 exhibits a commercial capsulating apparatus of the prior art for use in the capsulating process of the present invention.

FIG. 2 illustrates an apparatus for self-producing tablets.

FIGS. 3A, 3B, 3C and 3D are schematic representations of the basic steps in the apparatus for self-producing tablets.

### DETAILED DESCRIPTION OF THE INVENTION

The invention will now be described with reference to some non-limiting specific embodiments. The invention will first be illustrated in reference to the attached drawings to be followed by a more detailed description below.

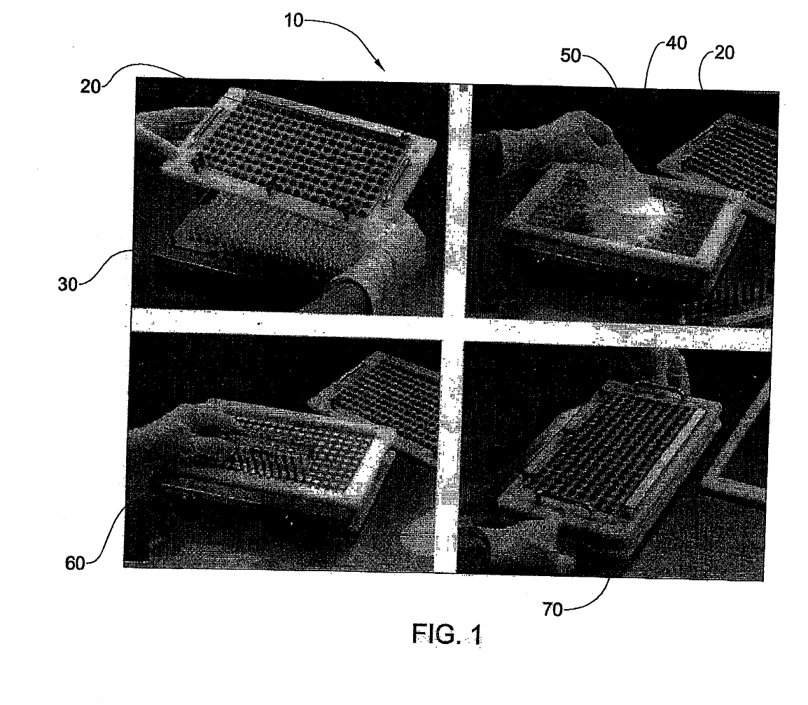

Reference is first being made to FIG. 1, which is a schematic representation of a commercial apparatus 10 for producing capsules described in a brochure provided by Feton®. Empty closed capsules, which are composed of lower and upper parts, are aligned in the apparatus. After the removal of the upper part 20 of the apparatus holding the upper parts of the capsules, the lower empty part of the capsules held by the lower part of the apparatus 30 is exposed. The upper part 20 is laid aside and a nutraceutical ingredient 40 is poured in the desired quantity and distributed evenly on all open lower parts of the capsules with the aid of a shovel 50. Tie nutraceutical ingredient in the lower parts of the filled capsules may be compressed with a “comb” like apparatus 60. In the next step the upper part of the apparatus 20 is returned to its location and the filled capsules are sealed 70 and released.

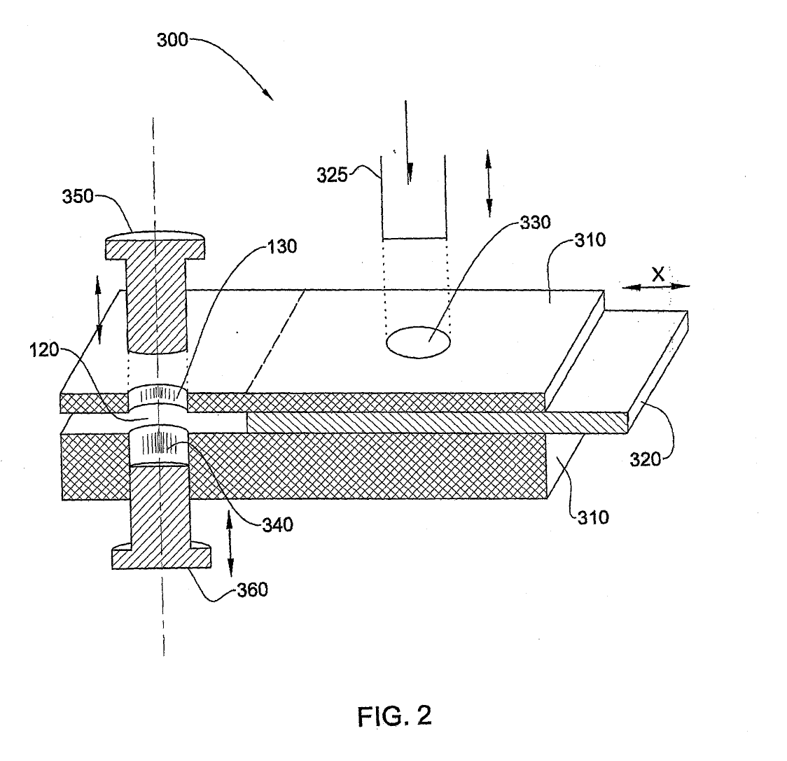

Reference is now being made to FIG. 2 illustrating the main constructional parts of an apparatus according to the invention for self-producing tablets, in its intermediate operational position. The apparatus 300 comprises a support structure 310 that carries a plate (or disc) 320 which is mounted for a reciprocating movement along X-axis inside a path 120 in the support structure or for a pivotal movement with respect to the support structure 310, and is formed with an opening 330 for a material to be received therein from an inlet vessel 325, when an alignment between the opening 330 and the vessel is provided; and has two vertically aligned recesses 340 of equal cross-sectional dimensions above and below the plate plane defining a compression path 130 for sliding movements of pistons 350 and 360 in the path 130 along the Y-axis towards and away from each other. The cross-sectional dimension of the recess 340 is equal to that of the opening 330. Preferably, the vessel 325 is also mounted for reciprocating along the Y-axis. It should be understood that when the plate 320 is located such that it is misaligned with the recess 340, the opening 330 is located above the surface of the structure and thereby forms a groove for supporting the material.

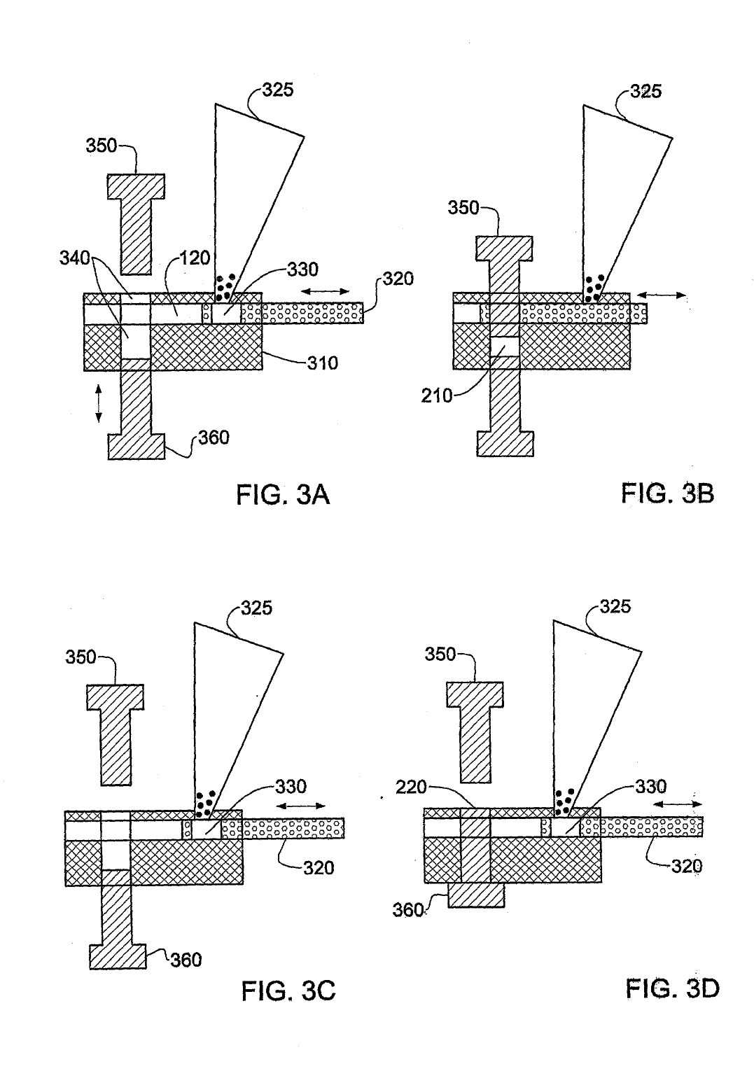

Reference is now being made to FIGS. 3A-3D representing the sequential operational steps carried out by the apparatus 300 for self-producing of tablets. Turing to FIG. 3A, at the initial operational step of the apparatus 300, where the support base 310 is brought to a location when the opening 330 is aligned with the distal end of the vessel 325, the vessel being appropriately located with its distal end being just above the groove 330. At this stage, the pistons 350 and 360 are in their upper and lower inoperative positions, respectively. The contents of the vessel comprising a required amount (dosage) of at least one nutraceutical ingredient optionally mixed with an excipient is discharged from the vessel into the groove 330. The plate 320 now moves inside the path 120 along the X-axis so as to align the opening 330 with the recesses 340.

At the next operational step, as shown in FIG. 3B, the upper piston 350 moves downwards along the recess 340 thereby moving the material from the opening towards the distal end of the lower piston 360, and compressing the material between the distal ends of the two pistons, thereby forming a tablet at 210.

Thereafter (FIG. 3C), the upper piston 350 rums back into its uppermost inoperative position, leaving the tablet to reside on the outer surface of the distal end of the lower piston 360, and the plate 320 returns back into its initial position shown in FIG. 3A. Turning to FIG. 3D, the lower piston 360 is moved upwards along the recess 340 thereby bringing the tablet from 210 to the upper surface of the structure 310, where it can be collected. The cycle of operations can then be repeated, where an amount of up to 250 tablets may be produced each hour.

As mentioned the present invention is directed to a “do-it-yourself” kit for production of personalized nutraceutical capsules and/or tablets. The advantage of such capsules and/or tablets from the individual's point of view is two-fold: reduction of the price, and the ability to produce and consume nutraceutical mixtures, which are not commercially available. The cost of the raw material for the production of nutraceutical capsules and/or tablets is 10-15% of the retail price and the costs of the packaging materials and direct labor present additional 10-15%. Marketing, promotion and distribution costs are responsible for the major part of the rest of the balance. Thus self-production of nutraceutical capsules and/or tablets give rise significant saving of money. In addition, commercial products frequently do not meet the individual needs and/or desire. An individual may want to formulate his/her own composition meeting his/her own bodily needs. The existing approach, at best, is in combining variable commercial capsules and/or tablets, which of course requires the consumption of many capsules and/or tablets. However, very frequently the nutraceutical products which the individual desires to consume are not necessarily available. From the producers point of view such an approach is also beneficial. A personalized approach for production of nutraceuticals would increase public awareness to the use of nutraceuticals in a daily diet, which ultimately result in a major increase in sales of the crude nutraceuticals. Furthermore, such an approach would also boost the nutraceutical market that seems as dropping into stagnation.

The instructions for the personalized production of the nutraceuticals according to the present invention include an operational manual, a guide for formulating of presently available products. In addition the instructions include a guide for formulating new products including safety aspects, detailed information on available raw material including its properties, health benefits, recommended usage levels, dosage limitations (where applicable), where all such information is continuously updated. Mail, email or telephone to retailers where raw materials can be purchased may all be used for updating. Alternatively updating may be done by contacting a given WEB address or by periodically updating by printed matter or electronic (CD) form. An individual may thus be able to utilize the kit of the present invention to easily produce a personalized product in small batches of ca. 50-200 capsules or tablets, wherein the capsules or tablets comprise a wide assortment of options to suit most of the individual nutraceutical needs.

Alternatively, the method of self-producing of tablets or capsules may be carried out in a different manner. A person desirous of obtaining personalized tablets or capsules comprising of at least one desired nutraceutical ingredient may submit the order to a local distributor. The distributor would then provide the desirous person with the appropriate instructions and required materials for such self-production.

For the case of self-production of capsules these include providing the desirous person with:

(a) the required amount of at least one nutraceutical ingredient;

(b) the required plurality of empty capsules;

(c) the appropriate apparatus for self-producing capsules;

(d) the required instructions including:

(i) pouring said at least one nutraceutical ingredient into said plurality of empty capsules, giving rise to filled capsules; and

(ii) closing said filled capsules, giving rise to personalized capsules.

For the case of self-production of tablets these include providing the desirous person with:

(a) the required amount of at least one nutraceutical ingredient;

(b) the required amount of at least one excipient;

(c) an appropriate apparatus for self-producing tablets;

(d) instructions required for self-producing said tablets with said apparatus comprising at least one nutraceutical ingredient including:

(i) pouring said at least one nutraceutical ingredient and at least one excipient into a feeder of a tableting apparatus;

(ii) pressing a predetermined quantity of said at least one nutraceutical ingredient together with at least one excipient in said tableting apparatus, giving rise to personalized tablet; and

(iii) repeating said pouring and pressing steps for a desired number of cycles giving rise to personalized tablets.

A typical capsule according to the invention may compose a quantity of from several hundreds of milligrams of nutraceutical ingredient, up to a quantity of about 1 gr. A typical tablet according to the invention may comprise a quantity of from several hundreds of milligrams of nutraceutical ingredient and excipient to a quantity of about 1.3 gr. The ratio of nutraceutical ingredient to excipient in the tablets is from about 60-90% of nutraceutical ingrediet and from about 10-40% of excipient.

The invention will now be illustrated by the following non-limiting examples.

### EXAMPLE 1

Device and Method for Producing Capsules

The kit consists of the following items: a. Device for aligning, opening, pressing and closing the caps. Such a device is obtained by the “down scaling” of a commercial manual capsule-filling machine. b. A small balance weighing accurately (±0.1 g) up to 100 grams. c. A small powder blender. d. Plastic scalp. e. Gloves. f. Designed containers for final product. g. Empty capsules (optional colors and sizes). h. Blank labels.

### EXAMPLE 2

Device and Method for Producing Tablets

A kit consisting of: a. Device for blending, pressing and extruding tablets. Such a device is as described above, i.e. an apparatus 300 of the present invention. b. A small balance weighing accurately (±0.1 g) up to 100 grams. c. Gloves. d. Designed containers for final product. e. A suitable excipient (such as micro crystaline cellulose). f. Blank labels.

### EXAMPLE 3

Preparation of Capsules

A. General

The appropriate formulation is chosen out of over 100 options, or any personal preference according to general instructions enclosed together with the kit of Example 1. The process for the preparation comprises the following steps: 1. Desired receipt is pulled out. 2. Raw materials from the bulk powder containers are weighed according to the number of capsules that should be prepared. 3. Using the blender, the powders are blended for the required period. 4. Aligning of empty capsules in the machine. 5. Opening the capsules using the head device. 6. Filling the open empty halves utilizing the scalp and the pressing device. 7. Placing the head device, pressing and removing the capsules to pre-labeled containers.

B. 200 Capsules comprising the combination of vitamin C and E are prepared. 81.6 gr of ascorbic acid (2% excess) and 51 gr of tocopherol (2% excess 0 are weighed and blended to yield a homogenous powder. 200 empty capsules are aligned in the appropriate apparatus, such as the one described above and the lower part of the capsules is exposed by lifting the upper part. The blended powder containing ascorbic and tocopherol is poured over the empty capsules and the capsules filled and compressed. Their upper part is returned, the capsules are sealed and released from the apparatus. The produced capsules are placed in a pre-labeled container ready for consumption.

### EXAMPLE 4

Preparation of Tablets

A. General

The appropriate formulation is chosen out of over 100 options, or any personal preference according to general instructions enclosed together with the kit of Example 2. The process for the preparation comprises the following steps: 1. Desired receipt is pulled out. 2. Raw materials from the bulk powder containers are weighed according to the number of capsules that should be prepared. 3. Placing the powders into the tableting machine chamber 325. 4. Operating the apparatus according to instructions. 5. Collecting the tablets into pre-labeled containers.

B. 200 tablets comprising vitamin C and fenugreek together with an appropriate excipient are prepared. 81.6 (2% excess) ascorbic acid, 124.5 gr (2% excess) fenugreek and 51.5 gr (2% excess) of MgStearate are weighed and blended to yield a homogenous powder. The homogenous powder is placed into the chamber of the apparatus of the invention and initiating the operation. The desired tablets are collected as formed and stored for consumption in a pre-labeled container.

1. A kit designed for self producing of personalized capsules containing at least one nutraceutical ingredient comprising:
(i) a system operable to
receiving user selected at least one nutraceutical ingredient, wherein said at least one nutraceutical ingredient is in a pre-defined quantity;
receiving plurality of empty capsules;
pouring said at least one nutraceutical ingredient into said plurality of empty capsules, giving rise to filled capsules; and
closing said filled capsules, giving rise to personalized capsules;
(ii) instructions for the preparation of the personalized capsules including designation of the pre-defined quantity of said at least one nutraceutical ingredient.

2. A kit designed for self producing of personalized tablets containing at least one nutraceutical ingredient comprising:
(i) a system operable to
receiving user selected at least one nutraceutical ingredient, wherein said at least one nutraceutical ingredient is in a pre-defined quantity;
receiving at least one excipient;
pouring said at least one nutraceutical ingredient and at least one excipient into a feeder of a tableting apparatus;
pressing a predetermined quantity of said at least one nutraceutical ingredient together with at least one excipient in said tableting apparatus, giving rise to personalized tablet; and
repeating said pouring and pressing steps giving rise to personalized tablets;
(ii) instructions for the preparation of the personalized tablets including designation of the pre-defined quantity of said at least one nutraceutical ingredient and said at least one excipient.

3. A kit according to claim 2, wherein said user selected at least one nutraceutical ingredient includes a pre-defined quantity of excipient.

4. A method for self producing of personalized capsules containing at least one nutraceutical ingredient, comprising:
providing instructions for the quantity required for producing a user selected capsule comprising at least one nutraceutical ingredient;
providing instructions how to self-produce personalized capsules, including:
(i) receiving plurality of empty capsules;
(ii) pouring said at least one nutraceutical ingredient into said plurality of empty capsules, giving rise to filled capsules; and
(iii) closing said filled capsules, giving rise to personalized capsules.

5. A method for self producing of personalized tablets containing at least one nutraceutical ingredient, comprising:
providing instructions for the quantity required for producing a user selected tablet comprising at least one nutraceutical ingredient;
providing instructions how to self-produce personalized tablets, including:
(i) receiving at least one excipient;
(ii) pouring said at least one nutraceutical ingredient and at least one excipient in a predefined quantity into a feeder of a tableting apparatus;
(iii) pressing a predetermined quantity of said at least one nutraceutical ingredient together with at least one excipient in said tableting apparatus, giving rise to personalized tablet; and
(iv) repeating said pouring and pressing steps for a desired number of cycles giving rise to personalized tablets.

6. A method according to claim 5, wherein said at least one nutraceutical ingredient comprises a pre-determined quantity of excipient.

7. A nutraceutical ingredient and associated instructions for use in the system of self-producing of personalized capsules as defined in claim 1.

8. At least one nutraceutical ingredient and associated instructions for use in a process of self producing of personalized capsules, the instructions comprising:
initial instructions for the maximum quantity per capsule of said at least one nutraceutical ingredient to use in the process; the process comprising:
(i) receiving plurality of empty capsules;
(ii) pouring said at least one nutraceutical ingredient into said plurality of empty capsules, giving rise to filled capsules; and
(iii) closing said filled capsules, giving rise to personalized capsules.

9. At least one nutraceutical ingredient and associated instructions for use in the system of self-production of personalized tablets as defined in claim 2.

10. At least one nutraceutical ingredient, excipient and associated instructions for use in the system of self-production of personalized tablets as defined in claim 3.

11. At least one nutraceutical ingredient at least one excipient and associated instructions for use in a process of self-producing of personalized tablets, the instructions comprising:
initial instructions for the maximum quantity per capsule of said at least one nutraceutical ingredient and said at least one excipient to use in the process; the process comprising:
(i) receiving at least one excipient;
(ii) pouring said at least one nutraceutical ingredient and at least one excipient into a feeder of a tableting apparatus;
(iii) pressing a predetermined quantity of said at least one nutraceutical ingredient together with at least one excipient in said tableting apparatus, giving rise to personalized tablet; and
(iv) repeating said pouring and pressing steps for a desired number of cycles giving rise to personalized tablets.

12. A method for producing of personalized capsules containing at least one nutraceutical ingredient comprising:
obtaining an order from a user desirous of producing capsules containing at least one desired nutraceutical ingredient;
providing the user with the required amount of at least one nutraceutical ingredient and with required plurality of empty capsules;
providing the user with an appropriate apparatus for self-producing capsules;
providing the user with instructions required for self-producing said capsules comprising at least one nutraceutical ingredient;
including:
(i) pouring said at least one nutraceutical ingredient into said plurality of empty capsules, giving rise to filled capsules; and
(ii) closing said filled capsules, giving rise to personalized capsules.

13. A method for self producing of personalized tablets containing at least one nutraceutical ingredient comprising:
obtaining an order from a user desirous of producing tablets containing at least one desired nutraceutical ingredient;
providing the user with the required amount of at least one nutraceutical ingredient and required at least one excipient;
providing the user with an appropriate apparatus for self-producing tablets providing the user with instructions required for self-producing said tablets with said apparatus comprising at least one nutraceutical ingredient;
including:
(i) pouring said at least one nutraceutical ingredient and at least one excipient into a feeder of a tableting apparatus;
(ii) pressing a predetermined quantity of said at least one nutraceutical ingredient together with at least one excipient in said tableting apparatus, giving rise to personalized tablet; and
(iii) repeating said pouring and pressing steps for a desired number of cycles giving rise to personalized tablets.

14. A system for self-producing tablets, the system comprising a support structure having an intermediate plate formed with an opening for receiving a required quantity of a material from which the tablet is to be produced, said plate being movable with respect to the structure along a horizontal path thereby providing displacement of said opening towards and away an elongated vertical recess so as to align the opening with the recess, the material, when in the aligned position of the opening being compressed into the tablet by mechanical pressure applied to the material from opposite sides thereof.
